(19) United States
(12) Patent Application Publication
(10) Pub. No.: US 2005/0175719 A1
(43) Pub. Date: Aug. 11, 2005

(54) Procyanidins for treatment and prevention of enzymatic irritation to the skin

(76) Inventors: Ying Sun, Belle Mead, NJ (US); Baozhen Lin, Belle Mead, NJ (US); Jue-Chen Liu, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/772,947

(22) Filed: Feb. 5, 2004

(51) Int. Cl. A61K 35/78

(52) U.S. Cl. 424/729; 424/765; 424/770; 424/766; 424/776; 424/642; 514/254.07; 514/63

(57) ABSTRACT

This invention relates to methods and compositions for preventing and treating skin rash, such as perineal dermatitis, associated with enzymatic dermatitis. More particularly, this invention relates to compounds containing procyanidins, which possess trypsin and/or chymotrypsin inhibitory activity and are suitable for use in compositions for preventing and treating skin irritation caused by protease exposure, such as perineal dermatitis.

### BACKGROUND OF THE INVENTION

1. Field of the Invention

The present invention relates to a topical composition for use in preventing and treating skin rash, such as perineal dermatitis, resulting from enzymatic irritation to the skin, and methods therefor.

2. Description of the Prior Art

One common skin rash is perineal dermatitis, which includes diaper dermatitis or “diaper rash.” Perineal dermatitis has been defined as contact dermatitis in the perineal area, including the perineum buttocks, and the perineal, coccyx, and upper/inner thigh regions. See Brown, D. S, et al., 39(7) A Conceptual Framework, Ostomy/Wound Management 20-25 (1993)(“Brown”). The physical signs of diaper dermatitis may include one or a combination of erythema, oozing, swelling, crusting, scaling, and visiculation, with the possibility of hyperpigmentation, thickening, and excoriation over time. See Brown.

Diaper dermatitis is believed to be caused by the prolonged contact of the skin with fecal matter and urine. Although the exact component or components of urine and feces responsible for diaper dermatitis has not been identified, some possible factors may include ammonia, urine pH, fecal microorganisms, and lipase and protease enzymes found in fecal matter.

Currently, the main focus of diaper dermatitis treatments has been to reduce the exposure of the skin to such body wastes via barrier products, e.g. diaper creams and lotions. However, such barrier products tend to only address the rash symptoms. It would be preferable to have a composition that not only would address diaper rash symptoms, but would also prevent the formation of the rash at the onset and/or the proliferation thereof.

Another mode of treatment focuses on the incorporation of agents to inhibit various fecal enzymes, such as lipase, that often aggravate perineal dermatitis. However, such agents are often synthetic in nature and, as such, may contribute to environmental pollution.

Therefore, there is a need for a composition that is effective at reducing, preventing, and/or treating perineal dermatitis such as diaper rash. Such compositions should not only be capable of effectively reducing and/or preventing the irritation caused by fecal enzymes, but also be biodegradable and environment-friendly.

### SUMMARY OF THE INVENTION

The present invention describes a method for treating and/or preventing enzymatic dermatitis, such as diaper rash, via the topical administration of trypsin and/or chymotrypsin inhibitors, including but not limited to procyanidin-containing products, or compositions containing such inhibitors, as defined in the claims.

Other features and advantages of the present invention will be apparent from the detailed description of the invention and from the claims

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified.

As used herein, “trypsin inhibitory activity” means the ability of the procyanidin-containing product, such as a plant extract, at a concentration of 0.01 (w/w) to inhibit the activity of the protease, trypsin, as measured by the assay set forth below in Example 1. In one embodiment, the OPC-containing products used in the present invention have a trypsin inhibitory activity of at least about 15%. In a further embodiment, the OPC-containing products used in the present invention have a trypsin inhibitory activity of at least about 25%, such as at least about 50% or at least about 60%.

As used herein, “chymotrypsin inhibitory activity” means the ability of the procyanidin-containing product, such as a plant extract, at a concentration of 0.01 (w/w) to inhibit the activity of the protease, chymotrypsin, as measured by the assay set forth below in Example 3. In one embodiment, the procyanidin-containing products used in the present invention have a chymotrypsin inhibitory activity of at least about 15%. In a further embodiment, the OPC-containing products used in the present invention have a chymotrypsin inhibitory activity of at least about 25%, such as at least about 50% or at least about 60%.

As used herein, “prevent” or “preventing” means to proactively stop the development of enzymatic skin irritation, such as perineal dermatitis and diaper rash, or to halt or slow down the progression of such irritation, or to reduce the risk of developing such irritation.

As used herein, “treat” or “treating” is meant to comfort the skin near and/or at the location of enzymatic skin irritation, such as diaper rash, and if possible to induce a regression in such irritation. As used herein, “regression” is meant to reduce the amount and/or severity of topical enzymatic skin irritation symptoms, such as, for example, irritation, redness, blisters, discomfort, excoriation, and the like.

As used herein, “topical application” means directly laying on or spreading on outer skin using, e.g., by use of the hands, an applicator such as a wipe, puff, roller, or spray, or via a substrate such as a diaper or sanitary napkin.

As used herein, “affected area” is meant the area of skin that is presently exhibiting any levels of skin rash or enzymatic dermatitis, or the area that will be in prolonged contact with feces containing such dermatitis-causing enzymes. This area also includes the area immediately proximate to the described area. It is the area at which treatment and/or prevention is desired.

As used herein, “cosmetically-acceptable” means that the product(s) or compound(s) which the term describes are suitable for use in contact with tissues (e.g., the skin) without undue toxicity, incompatibility, instability, irritation, allergic response, and the like.

As used herein, “topical carrier” means one or more compatible solid or liquid filler diluents that are suitable for topical administration to a mammal. Examples of topical carriers include, but are not limited to, water, waxes, oils, emollients, emulsifiers, thickening agents, gelling agents, and mixtures thereof.

As used herein, “cleansing” means the removal of dirt and/or oil from the skin, hair, or nail surface.

As used herein, “safe and effective amount” means an amount of compound or composition (e.g., OPC-containing product) sufficient to induce a positive modification in the condition to be regulated or treated, but low enough to avoid serious side effects. The safe and effective amount of the compound or composition will vary with the particular condition being treated, the age and physical condition of the end user, the severity of the condition being treated/prevented, the duration of the treatment, the nature of other treatments, the specific compound or product/composition employed, the particular cosmetically-acceptable carrier utilized, and like factors.

The topical compositions of the present invention comprise a safe and effective amount of the trypsin and/or chymotrypsin inhibitors (e.g. procyanidin-containing products). In one embodiment, the composition contains, based upon the total amount of topical composition, from about 0.001% to about 50%, e.g., from about 0.001% to about 30%, from about 0.01% to about 10% or from about 0.1% to about 1.0%, of the trypsin and/or chymotrypsin inhibitors (e.g., procyanidin-containing products).

Procyanidins

As used herein, “procyanidin” or “procyanidolic oligomers” or “proanthocyanidins” or “OPC” shall mean the linear or branched polyphenolic oligomer compounds containing about 15 or less, e.g., about 10 or less or about 8 or less, monomer units in their backbone, and derivatives thereof, such as esters. Examples of such monomer units include, but are not limited to catechin, epicatechin, catechin gallate, epicatechin gallate, gallocatechin gallate, and epigallocatechin gallate.

In one embodiment, the procyanidins have an average molecular weight of about 300 to about 7,000, e.g., from about 600 to about 2,500.

Suitable procyanidins for use in the present invention are those which are members of the flavonoid family and which may be obtained from natural plant sources, which include but are not limited to grape seeds, pine barks such as the pine bark extract available from Henkel Nutrition and Health Group under the tradename, “Pycnogenol”, pine buds, apples such as the apple cider therefrom, tea such as green tea, cocoa, saxifraga stolonifera which is commercially available from Ichimura Pharcos Co. Ltd. under the tradename “Yukinoshita”, and the like. Examples of suitable procyanidins include those disclosed in WO 01/41775.

Topical Compositions

The topical compositions useful in the present invention involve formulations suitable for topical application to skin. In one embodiment, the composition comprises an OPC-enriched plant extract product and a cosmetically acceptable topical carrier. In one embodiment, the composition contains the cosmetically acceptable topical carrier in an amount, based upon the total weight of the composition, from about 50% to about 99.99%, e.g., from about 80% to about 95%.

In one embodiment the topical composition is substantially free of isocourmanns or courmarins, i.e., e.g., contains less than about 1% or less than about 0.1% or less than about 0.01%.

The topical compositions may be made into a wide variety of product types that include but are not limited to lotions, creams, gels, sticks, sprays, shaving creams, ointments, cleansing liquid washes and solid bars, shampoos, pastes, powders, mousses, wipes, patches, nail lacquers, wound dressing and adhesive bandages, hydrogels, films, and make-up such as foundations, mascaras, and lipsticks. These product types may comprise several types of cosmetically acceptable topical carriers including, but not limited to solutions, emulsions (e.g., microemulsions and nanoemulsions), gels, solids and liposomes. The following are non-limitative examples of such carriers. Other carriers can be formulated by those of ordinary skill in the art.

The topical compositions useful in the present invention can be formulated as anhydrous products. As used herein, “anhydrous” shall mean that the compositions contain less than about 10 percent, e.g., less than about 5 percent or less than about 1 percent of water.

Alternatively, the topical compositions useful in the present invention can be formulated as solutions. Solutions typically include water (e.g., from about 50% to about 99.99% or from about 90% to about 99% of water).

Topical compositions useful in the subject invention may be formulated as a solution comprising an emollient. Such compositions may contain from about 2% to about 50% of an emollient(s). As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972) and the International Cosmetic Ingredient Dictionary and Handbook, eds. Wenninger and McEwen, pp.1656-61, 1626, and 1654-55 (The Cosmetic, Toiletry, and Fragrance Assoc., Washington, D.C., 7^{th} Edition, 1997) (hereinafter “CI Handbook”) contain numerous examples of suitable materials.

A lotion can be made from such a solution. Lotions typically comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient(s) plus from about 0.1% to about 2% of a thickening agent(s). A more complete disclosure of thickening agents or viscosity increasing agents useful herein can be found in Sagarin, Cosmetics, Science and Technology, 2nd Edition, Vol. 1, pp. 72-73 (1972) and the ICI Handbook pp. 1693-1697. In one embodiment, the ointment is anhydrous. In another embodiment, the procyanidin-containing products are solubilized or dispersed in the lipophilic phase of the ointment.

The topical compositions useful in the present invention can also be formulated as emulsions. If the carrier is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, U.S. Pat. No. 3,755,560, U.S. Pat. No. 4,421,769, McCutcheon's Detergents and Emulsifiers, North American Edition, pp. 317-324 (1986), and the ICI Handbook, pp.1673-1686.

Lotions and creams can be formulated as emulsions. Typically such lotions comprise from 0.5% to about 5% of an emulsifier(s). Such creams would typically comprise from about 1 % to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the cosmetic art and are useful in the subject invention. Multiphase emulsion compositions, such as the water-in-oil-in-water type, as disclosed in U.S. Pat. No. 4,254,105 and 4,960,764, are also useful in the subject invention. In general, such single or multiphase emulsions contain water, emollients, and emulsifiers as essential ingredients.

The topical compositions of this invention can also be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprises between about 0.1% and 5%, by weight, of such gelling agents.

The topical compositions of this invention can also be combined with particulates such as clay, silica, talc, starch such as cornstarch, and the like, and optional flow agents such as tricalcium phosphate, in order to form dusting powders. Examples of such powder components and methods for making powder compositions may be found in, for example, U.S. Pat. Nos. 4,568,539 and 4,485,092.

The topical compositions of the present invention can also be formulated into a solid formulation (e.g., a wax-based stick, soap bar composition, or a wipe containing powder).

Liposomal formulations are also useful compositions of the subject invention. Examples of liposomes are unilamellar, multilamellar, and paucilamellar liposomes, which may or may not contain phospholipids. Such compositions can be prepared by first combining hesperetin with a phospholipid, such as dipalmitoylphosphatidyl choline, cholesterol and water according to the method described in Mezei & Gulasekharam, “Liposomes—A Selective Drug Delivery System for the Topical Route of Administration; Gel Dosage Form”, Journal of Pharmaceutics and Pharmacology, Vol. 34 (1982), pp. 473-474, or a modification thereof. Epidermal lipids of suitable composition for forming liposomes may be substituted for the phospholipid. The liposome preparation may then incorporated into one of the above carriers (e.g., a gel or an oil-in-water emulsion) in order to produce the liposomal formulation. Other compositions and pharmaceutical uses of topically applied liposomes are described in Mezei, M., “Liposomes as a Skin Drug Delivery System”, Topics in Pharmaceutical Sciences (D. D. Breimer and P. Speiser, eds.,), Elsevier Science Publishers B. V., New York, N.Y., 1985, pp. 345-358, PCT Patent Application No. WO96/31194 and U.S. Pat. No. 5,260,065.

The topical compositions useful in the subject invention may contain, in addition to the aforementioned components, a wide variety of additional oil-soluble materials and/or water-soluble materials conventionally used in compositions for use on skin at their art-established levels.

Additional Cosmetically Active Agents

In one embodiment, the topical composition further comprises another cosmetically active agent in addition to the procyanidin-containing product. What is meant by a “cosmetically active agent” is a compound that has a cosmetic or therapeutic effect on the skin, hair, or nails, e.g., lightening agents, darkening agents such as self-tanning agents, anti-acne agents, shine control agents, anti-microbial agents, anti-inflammatory agents, anti-mycotic agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, firming agents, anti-callous agents, anti-diaper rash agents, and other agents for hair, nail, and/or skin conditioning.

In one embodiment, the agent is selected from, but not limited to, the group consisting of hydroxy acids, benzoyl peroxide, sulfur resorcinol, ascorbic acid, D-panthenol, hydroquinone, octyl methoxycinnimate, titanium dioxide, octyl salicylate, homosalate, avobenzone, polyphenolics, carotenoids, free radical scavengers, spin traps, retinoids such as retinol and retinyl palmitate, ceramides, polyunsaturated fatty acids, essential fatty acids, enzymes, enzyme inhibitors such as the soy trypsin inhibitor disclosed in U.S. Pat. No. 6,555,143, minerals, hormones such as estrogens, steroids such as hydrocortisone, 2-dimethylaminoethanol, copper salts such as copper chloride, peptides containing copper such as Cu:Gly-His-Lys, coenzyme Q10, peptides such as those disclosed in PCT Patent Application WO00/15188, lipoic acid, amino acids such a proline and tyrosine, vitamins, lactobionic acid, acetyl-coenzyme A, niacin, riboflavin, thiamin, ribose, electron transporters such as NADH and FADH2, and other botanical extracts such as aloe vera, non-denatured soy extract such as that disclosed in U.S. Pat. No. 6,155,143, feverfew extracts and derivatives and mixtures thereof. The cosmetically active agent will typically be present in the composition of the invention in an amount of from about 0.001% to about 20% by weight of the composition, e.g., about 0.01% to about 10% such as about 0.1% to about 5%.

In one embodiment, the anti-diaper rash agent is one or more of the following: zinc oxide, antifungals such as ketoconazole, miconazole, elubiol, allantoin, calamine, dimethicone, kaolin, petrolatum, white petrolatum, cod liver oil, lanolin, mineral oil, talc, topical starch, and any other agent suitable for use in the treatment and/or prevention of diaper rash.

In another embodiment, the anti-diaper rash agent is one or more of the following agents in an amount, based upon the total weight of the composition, from about 0.5% to about 2% of allantoin, from about 1% to about 25% calamine, from about 1 % to about 30% dimethicone, from about 4% to about 20% kaolin, from about 30% to less than about 100% petrolatum, from about 30% to less than about 100% of white petrolatum, from about 5% to about 14% of cod liver oil, e.g. such that the amount of cod liver oil does not exceed 10,000 USP units of vitamin A and 400 USP units of cholecalciferol, about 10% to about 16% of lanolin, from about 50% to less than about 100% mineral oil, from about 45% to less than about 100% talc, from about 10% to about 87% topical starch, and from about 35% to about 40% zinc oxide. Examples of vitamins include, but are not limited to, vitamin A, vitamin Bs such as vitamin B3, vitamin B5, and vitamin B12, vitamin C, vitamin K, and vitamin E and derivatives thereof.

Examples of hydroxy acids include, but are not limited, to glycolic acid, lactic acid, malic acid, salicylic acid, citric acid, and tartaric acid. See, e.g., European Patent Application No. 273,202.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, and propolis. Other examples of antioxidants may be found on pages 1612-13 of the ICI Handbook. In embodiments wherein a procyanidin-containing natural extract is added to the composition for antioxidant properties, it shall be understood that such extract shall be added in an amount that exceeds the upper range of the amount used for the trypsin/chymotrypsin inhibitory property.

Other Materials

Various other materials may also be present in the topical compositions useful in the subject invention. These include humectants, proteins and polypeptides, preservatives and an alkaline agent. Examples of such agents are disclosed in the ICI Handbook, pp.1650-1667.

The topical compositions of the present invention may also comprise chelating agents (e.g., EDTA) and preservatives (e.g., parabens). Examples of suitable preservatives and chelating agents are listed in pp. 1626 and 1654-55 of the ICI Handbook. In addition, the topical compositions useful herein can contain conventional cosmetic adjuvants, such as dyes, opacifiers (e.g., titanium dioxide), pigments, and fragrances.

The procyanidin-containing topical compositions of the present invention may be prepared using methodology that is well known by an artisan of ordinary skill. See, e.g., WO/41775.

Effective amounts of the topical compositions of the present invention have unexpectedly shown to be useful in the treatment and prevention of skin rash, such as diaper rash, resulting from enzymatic irritation to the skin. Without wishing to be bound by theory, it is believed that the trypsin/chymotrypsin inhibitory property of the procyanidin-containing product contributes to reduce the activity of proteases, such as the proteases from fecal material that may possibly cause the diaper rash itself (e.g. trypsin and/or chymotrypsin), and thus alleviates the skin disorder. Although the “effective amount” of topical composition used to treat or prevent the rash will depend upon, for example, the severity of skin irritation at the affected area, the concentration of enzymes in the exudate, the concentration of procyanidins in the composition, and the like, typically for treating or preventing purposes, from about 0.1 mg/square cm to about 3 mg/square cm, for example from about 1 mg/square cm to about 2.5 mg/square cm of the topical composition may be applied to the affected area.

The compositions of the present invention may be applied directly to the affected area of the skin or via a substrate such as a wipe, diaper liner, sanitary napkin, or facing to such affected area.

Although the structure of the substrate is not critical to the practice of the present invention, examples of suitable substrates are disclosed in, for example, U.S. Pat. No. 6,204,596 and WO99/26619.

The substrate may have the composition incorporated therein, or alternatively the composition may be provided separately from the substrate and applied thereto at the time of use. The amount of composition incorporated into the substrate is an amount effective for delivering the required treatment, reduction and/or prevention of the dermatitis. In one embodiment, the substrate contains the composition at such a level that the composition is present therein at a level of, based upon the total weight of the substrate, from about 35 g/square meter to about 90 g/square meter for example from about 50 g/square meter to 75 g/square meter.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLES

### Example 1

### Trypsin Inhibitory Activity of Procyanidins

The inhibition of trypsin-induced cleavage of a fluorescent casein peptide was measured using the EnzChek™ protease assay kit, following manufacturer's instructions (EnzChek™ Protease Assay Kits Product Information, Revised Mar. 15, 1999; Molecular Probes, Eugene Oreg.). In summary, various OPC preparations were first diluted in 1× digestion buffer (provided in assay kit) and incubated at different concentrations with 100 units of trypsin (Sigma, St. Louis, Mo.) dissolved in 1× digestion buffer. A pure serine protease inhibitor (soybean trypsin inhibitor or “STI”, from Sigma, St. Louis, Mo.) was used as a positive control at 0.1, 0.01%, and 0.001% w/v. Then, 1.0 mg/ml stock solution of BODIPY FL casein was prepared by adding 0.2 mL of deionized water to the vials supplied with this substrate (provided in assay kit), then made to a final working concentration of 10 microgram/ml in 1× digestion buffer.

Following incubation of the trypsin, with or without the test material, with the BODIPY fluorescent casein substrate at room temperature for one hour, fluorescence was measured (excitation 485 nm/emission 530 nm) using a SpectraMax® Gemini microtiter plate reader (Molecular Devices Corporation, Sunnyvale, Calif.) using Softmax® Pro 3.0 software (Molecular Devices Corporation), and % inhibition of trypsin was calculated. Each experiment was performed in three replicates and was repeated twice for each procyanidin containing preparation.

The above experiments were then independently repeated using various STI preparations and antioxidant preparations in order to measure the inhibition of trypsin-induced cleavage.

The percent inhibition of trypsin cleavage of the substrate by the procyanidin-containing preparations relative to that of preparations containing STI or commonly used antioxidants was calculated using Microsoft Excel™ and the results were summarized in Table 1 below.

TABLE 1
Trypsin Inhibitory Activity of Procyanidin-Containing
Compound, STI, and Antioxidants

| Concentrations | % trypsin inhibition | | | | |
|---|---|---|---|---|---|
| % (w/w) | OPC* | STI | Pycnogenol** | Vit C | Lactoferrin |
| 0 | 0 | 0 | 0 | 0 | 0 |
| 0.0005 | 15 | 17 | | | |
| 0.001 | 24 | 30 | | −2 | −2 |
| 0.0025 | 40 | 44 | | | |
| 0.005 | 57 | 55 | 38 | | |
| 0.075 | 65 | 63 | | | |
| 0.01 | 70 | 74 | 60 | 1 | −4 |
| 0.025 | 81 | 85 | | | |
| 0.05 | 83 | 87 | 88 | −12 | |
| 0.1 | | | | | 0 |

*the procyanidin-containing compound was extracted from grape seeds and was available from MMP Inc., under the tradename, “OPC From Grape Seeds”
**pine bark extract available from Henkel Nutrition and Health Group., under the tradename, “pycnogenol”.

This Example showed that the inhibitory activity of procyanidin-containing grape seed extract against the trypsin protease was comparable to that of a pure serine protease inhibitor (e.g. soybean trypsin inhibitor, STI). The results further showed that the trypsin inhibitory activity of this extract was not related to its antioxidant activity because other commonly used antioxidants e.g., Vitamin C and lactoferrin, had no trypsin inhibition activity at concentrations up to 0.1%.

### Example 2

### Trypsin Inhibitory Activity of Procyanidins

The procedure set forth in Example 1 was repeated, but with the substitution of other procyanidin enriched natural extracts, independently, for the grape seed extract used in Example 1.

The percent inhibition of trypsin cleavage of the substrate by these procyanidin enriched extracts were summarized in Table 2-1 and Table 2-2 below:

TABLE 2-1
Liquid Extracts: Trypsin Inhibitory Activity of Green Tea
Liquid Extracts, Pine Bud Liquid Extract, apple cider and apple juice

| Concen- | % trypsin inhibition | | | | | |
|---|---|---|---|---|---|---|
| trations | Excyte* | Incyte* | Phocyte | | | |
| % (v/v) of | green | Green | * green | Pine | Apple | Apple |
| Sample | tea | Tea | tea | Bud** | Cider*** | Juice**** |
| 0 | 0 | 0 | 0 | 0 | 0 | 0 |
| 1 | 90 | | | | | |
| 2 | | 51 | 69 | 6 | | |
| 5 | | 71 | 94 | 22 | | |
| 10 | | 82 | 105 | 43 | | |
| 20 | | | | 71 | | |
| 50 | | | | 88 | 50 | 24 |
| 100 | | | | 95 | 75 | 36 |

*available from Collaborative Labs
**available from Bioland Ltd.
***available from M. H. Ziegler & Sons, Inc.
****available from Wakefem Food Corp.

TABLE 2-2
Solid Extracts: Trypsin Inhibitory Activity of Green Tea
Extracts and Saxifraga Stolonifera

| | % trypsin inhibition | | | |
|---|---|---|---|---|
| Concen- | | GTE | | |
| trations | | Muktar's | 4864 | Green tea |
| % (v/w) | Saxifraga | green tea | green tea | extract 75% |
| of Samples | Stolonifera * | extract ** | extract *** | CG **** |
| 0 | 0 | 0 | 0 | 0 |
| 0.005 | 48 | 38 | 36 | 53 |
| 0.05 | 108 | 82 | 75 | 102 |

* Saxifraga Stolonifera Extract is commercially available from Ichimaru Pharcos Co. Ltd. under the tradename, “Yukinoshita”
** available from Univ. of Michigan Medical Center.
*** available from Provital, S.A.
**** available from Sabinasa Corporation.

This Example showed that procyanidin enriched natural extracts, such as green tea extracts, pine bud extract, Saxifraga Stolonifera extract, and apple extracts, exhibited inhibitory activity against the trypsin protease.

### Example 3

### Chymotrypsin/Acid Protease Inhibitory Activity of Procyanidins

The procedure of Example 1 was repeated for three OPC-containing grape seed extract formulations, but with the substitution of 5.5 units of chymotrypsin (type II, from bovine pancrease (Sigma, St. Louis, Mo.)) or 4 units of a thermolysin acid protease, which is commercially available as “protease X from Bacillus Thermoproteolyticus,” (Sigma, St. Louis, Mo.) for the 100 units of trypsin (Sigma, St. Louis, Mo.).

The percent inhibition of chymotrypsin and thermolysin cleavage against the substrate by the procyanidin containing preparations were calculated using Microsoft Excel™ and summarized in Table 3 below.

TABLE 3

| | % Trypsin Inhibition | | |
|---|---|---|---|
| Proteases | 0.001* | 0.01* | 0.1* |
| trypsin | 23 | 76 | 93 |
| α-Chymotrypsin | 19 | 57 | 85 |
| Thermolysin | | 40 | 59 |

*procyanidin-containing Grape Seed Extract Sample concentration % (w/v)

This Example further demonstrated that the procyanidin-containing grape seed extract had a significant dose-dependent inhibitory activity against serine proteases (trypsin and chymotrypsin) as well as an effective dose-dependent inhibitory activity against acid protease (thermolysin).

### Example 4

### Trypsin Inhibitory Activity of OPC, STI, and Monomer Units

The procedure of Example 1 was independently repeated using the procyanidin-containing grape seed extract and STI, respectively, at 0.1% concentration. In addition, the procedure of Example 1 was repeated on the five individual monomer units typically found in OPC, at up to three different concentration levels.

The percent inhibition of trypsin cleavage of the substrate by the procyanidin preparations relative to that of preparations containing STI or each of the five monomer units were calculated using Microsoft Excel™ and were summarized in Table 4 below.

TABLE 4

| | % trypsin inhibition | | |
|---|---|---|---|
| Concentration (w/w) (%) | 0.1 | 1 | 2.5 |
| Soybean trypsin inhibitor | 89 | | |
| Grape Seed Extract Containing | 82 | | |
| OPC having less than about 8 | | | |
| mer units | | | |
| Catechin (+/−) | 4.6 | 61 | 76 |
| Epicatechin (+) | 22 | | |
| Epicatechin (−) | | 71 | 90 |
| Catechin (+) | 11 | 72 | 89 |
| Catechin (−) | 18 | 70 | 92 |

This Example demonstrated that the five major monomer units of OPC also inhibited trypsin activity in a dose-responsive manner, although the individual monomer units possessed a relatively lower trypsin inhibitory activity in comparison to that obtained when they were polymerized into an OPC oligomer.

### Example 5

### Treatment and Prevention of Diaper Rash

After cleaning the perineal area of skin affected by diaper rash with a wipe available from JOHNSON & JOHNSON, Ltd., under the tradename, “JOHNSON's BABY Skincare” wipes, approximately 1 g of an ointment containing about 0.5% of the procyanidin-containing grape seed extract of Example 1 is topically applied to that area of skin. The area is then covered with a clean diaper.

Approximately two (2) hours later, the diaper is removed, and the same perineal skin area is visually observed. The affected area visually appears to have reduced surface area, and the redness of the skin in the affected area is reduced.

This Example shows that the OPC-containing product of the present invention is effective in preventing and treating diaper rash.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. A method of treating skin rash resulting from enzymatic irritation to the skin comprising administering to a mammal a composition containing an effective amount of a plant extract, wherein the plant extract has a trypsin and/or chymotrypsin inhibitory activity of at least about 15%.

2. The method according to claim 1 wherein said plant extract is comprised of a procyanidin.

3. The method according to claim 2 wherein said plant extract is available from at least one of the following natural plant sources: grape seeds, pine barks, pine buds, apples, saxifraga stolonifera, tea, or cocoa.

4. The method according to claim 3 wherein said plant extract is obtained from grape seeds.

5. The method according to claim 2 wherein the plant extract is a “Pycnogenol” pine bark extract.

6. The method according to claim 1 wherein the effective amount of the plant extract is, based upon the total weight of the composition, from about 0.01 percent to about 10 percent.

7. The method of claim 1 wherein the skin rash is perineal dermatitis.

8. The method of claim 7 wherein the composition is further comprised of at least one of the following anti-diaper rash agents: zinc oxide, ketoconazole, miconazole, elubiol, allantoin, calamine, dimethicone, kaolin, petrolatum, white petrolatum, cod liver oil, lanolin, mineral oil, talc, or topical starch.

9. The method of claim 7 wherein the composition is further comprised of a non-denatured soybean extract.

10. The method of claim 7, wherein the composition is in an anhydrous form.

11. The method of claim 7, wherein the composition is in the powder or ointment form.

12. A method of treating perineal dermatitis comprising topically administering to a mammal a composition containing, based upon the total weight of the composition, from about 0.01% to about 10% of a procyanidin-containing natural plant extract, wherein the plant extract has a trypsin and/or chymotrypsin inhibitory activity of at least about 15%.

13. The method of claim 12, wherein the plant extract is obtained from at least one of the following plant sources: grape seeds, pine barks, pine buds, apples, saxifraga stolonifera, tea, or cocoa.

14. A method of preventing diaper rash comprising topically administering to a mammal a composition containing, based upon the total weight of the composition, from about 0.01% to about 10% of an effective amount of a procyanidin-containing natural plant extract, wherein the plant extract has a trypsin and/or chymotrypsin inhibitory activity of at least 15%.

15. The method according to claim 14 wherein said procyanidin-containing natural plant extract is obtained from at least one of the following natural plant sources: grape seeds, pine barks, pine buds, apples, saxifraga stolonifera, tea, or cocoa.

16. The method according to claim 15 wherein said natural plant source is grape seeds.

17. The method according to claim 14 wherein the procyanidin-containing plant extract is “Pycnogenol” pine bark extract.

18. The method according to claim 14 wherein the effective amount of the procyanidin-containing plant extract is, based upon the total weight of the composition, from about 0.1 percent to about 1 percent.

19. The method according to claim 14 wherein the composition is further comprised of at least one of the following anti-diaper rash agents: zinc oxide, ketoconazole, miconazole, elubiol, allantoin, calamine, dimethicone, kaolin, petrolatum, white petrolatum, cod liver oil, lanolin, mineral oil, talc, or topical starch.

20. The method of claim 14 wherein the composition is further comprised of a non-denatured soybean extract.

21. The method of claim 14, wherein the composition is in an anhydrous form.

22. The method of claim 14, wherein the composition is in the powder or ointment form.

23. A method of preventing perineal dermatitis comprising topically administering to a mammal an anhydrous composition containing, based upon the total weight of the composition, from about 0.01% to about 10% of a procyanidin-containing natural plant extract, wherein the plant extract has a trypsin and/or chymotrypsin inhibitory activity of at least aboutl 5%.

24. The method of claim 23, wherein the procyanidin-containing plant extract is obtained from at least one of the following plant sources: grape seeds, pine barks, pine buds, apples, saxifraga stolonifera, tea, or cocoa.

25. A method of reducing the redness of skin caused by perineal dermatitis comprising topically administering to a mammal an anhydrous composition containing, based upon the total weight of the composition, from about 0.01% to about 10% of a procyanidin-containing natural plant extract, wherein the natural plant extract has a trypsin and/or chymotrypsin inhibitory activity of at least about 15%.

26. A method of treating skin rash resulting from enzymatic irritation to the skin comprising administering to a mammal a composition containing an effective amount of a natural plant extract having a trypsin and/or chymotrypsin inhibitory activity of at least 15%, wherein the natural plant extract is comprised of at least one of the following: procyanidin, catechin, or epicatechin.

27. A composition comprising, based upon the total weight of the composition,
a) from about 0.01% to about 10% of a procyanidin-containing natural plant extract, wherein the plant extract has a trypsin and/or chymotrypsin inhibitory activity of at least about 15%; and
b) at least one of the following cosmetically active agents: zinc oxide and/or non-denatured soy extract.
